(19) United States
(12) Patent Application Publication
(10) Pub. No.: US 2024/0293008 A1
(43) Pub. Date: Sep. 5, 2024

(54) SYSTEMS, DEVICES AND METHODS FOR ATTACHING ACCESSORY COMPONENTS TO ENDOSCOPIC INSTRUMENTS

(71) Applicant: GI SCIENTIFIC, LLC, Arlington, VA (US)

(72) Inventor: Scott Miller, Arlington, VA (US)

(73) Assignee: GI SCIENTIFIC, LLC, Arlington, VA (US)

(21) Appl. No.: 18/659,410

(22) Filed: May 9, 2024

Related U.S. Application Data
(63) Continuation of application No. PCT/US2022/049068, filed on Nov. 6, 2022.
(60) Provisional application No. 63/277,639, filed on Nov. 10, 2021.
(60) Provisional application No. 63/277,777, filed on Nov. 10, 2021.

(51) Int. Cl. A61B 1/00; A61B 1/018

(52) U.S. Cl. CPC A61B 1/0014; A61B 1/00043; A61B 1/018

(57) ABSTRACT

Systems, devices and methods are provided for coupling an accessory device, such as a disposable coupler, to an endoscopic instrument, such as an endoscope. A loading device includes a main body or base element with a proximal portion configured to receive the endoscope and a distal portion configured to receive the accessory device. The main body or base element allows an operator to stabilize the delivery area of the endoscope in a predictable, consistent manner. The loading device may further include one or more fixation elements to stabilize the distal end of the endoscope, preventing it from flexing and articulating during delivery of the accessory device onto the endoscope. A system may further include a delivery device or insertion tool for advancing the accessory device into the channel of the loading device.

### CROSS-REFERENCE TO RELATED APPLICATIONS

This application is a continuation of International Application No. PCT/US2022/049068 filed Nov. 6, 2022, which claims priority to U.S. Provisional Application No. 63/277,639 filed Nov. 10, 2021, and U.S. Provisional Application No. 63/277,777 filed Nov. 10, 2021, the entire contents are hereby incorporated by reference in their entirety.

### BACKGROUND

Recent advances in optical imaging technology have allowed many medical procedures to be performed in a minimally invasive manner. The evolution of sophisticated, flexible endoscopes with advanced visual capabilities has allowed access to regions deep within the human body that could only be achieved before with invasive surgical intervention. This modern day convenience has increased demand for, as well as the number of, endoscopic, laparoscopic, arthroscopic, ophthalmoscopic, borescopic, or other remote imaging visualization procedures performed every year in the U.S.

Endoscopy, for instance, is a procedure in which an endoscope is inserted into the patient's body for the visual examination of the interior of the body, including lumens, a body cavity or various organs like the heart, liver, pancreas, stomach, colon, bladder, reproductive systems and other parts of the body. Typical endoscopes are long and slender, with a lighted optical feature, to allow ease of introduction into the patient as well as visualization in the process. Endoscopes can be used with other medical devices, instruments or components, such as for example, cutting or cauterizing instruments, for treatment in addition to examination of the body cavity or organ.

Recently, devices which serve as convenient accessories for currently existing endoscopes have been used for a variety of reasons to improve patient care, especially with respect to accessories on the distal end of the endoscope. This includes devices used for aiding in tissue dissection (such as hollow caps), bleeding control (such as variceal banding devices), removable caps to provide more direct access to clean certain areas, disposable endoscopic shields that seal the distal end of the endoscope, hollow caps placed on the end of colonoscopes and gastroscopes that are used to help assess the depth of field in certain cases when in the patient, and other accessories. Some of these endoscopic companion devices or components can be placed over, or onto, or attached to, the endoscope, and include but are not limited to, endoscopic caps, endoscopic shields, sheaths, optical couplers, lenses, variceal banding devices, and endoscopic submucosal resection devices, to name a few. These devices further enhance the functionality of the endoscope and/or provide protection from contamination.

One example of such an accessory device is a removable coupler device that may be attached to the working end of an endoscope to reduce the risk of contamination and infection. For example, U.S. Pat. No. 10,856,724, the entirety of which is incorporated herein by reference for all purposes, describes an endoscope accessory for attaching to a working end of an endoscope that allows the user to simultaneously protect the working end of the endoscope from bacterial contamination by reducing the ingress of debris, fluid, bacteria, and other unwanted matter, while also enabling instruments to exit out of the working end of the endoscope at different angles with ease.

A common issue with delivering these accessories is inconsistent delivery or loading of these devices on to the endoscope. With the exception of endoscopic shields, which come with a custom delivery system, all of these accessories are loaded on to an endoscope by hand, using unguided manual manipulation and pressure from a healthcare worker, such as a nurse or technician, to place the accessory on the distal end of the endoscope. None of these accessories, other than endoscopic shields, have any technology providing specific guidance during delivery, any confirmatory delivery element, engineered alignment features, or other attributes which aid and confirm effective delivery on to an endoscope. Consequently, delivery is variable and leaves the patient at risk of an adverse event if delivery of these accessories does not occur effectively.

These adverse events are not a hypothetical concern. Multiple adverse events are reported each month to the FDA that detail circumstances in which an accessory was not properly delivered on to an endoscope resulting in patient injury. These injuries include accessories coming off in the middle of a case and blocking the patient's airway, creating a catastrophic circumstance for the patient. Other reported adverse events include an accessory not fully covering part of the distal end of the endoscope resulting in tissue injury to the patient, including stomach laceration, esophageal injury, and gastrointestinal tract perforations. Other issues result in a removal of the endoscope to fix the accessory issue and then reintroduction of the endoscope into the patient, which can create additional complications, including tissue trauma and complications related to extending the sedation time for the patient under anesthesia

Accordingly, it is desirable to provide devices that facilitate the attachment of accessory or companion devices to existing endoscopic devices, such as endoscopes, to remove the operator variability from this procedure and potentially reduce adverse outcomes for patients.

### SUMMARY

Systems, methods and devices are disclosed herein for attaching, delivering or loading accessory devices to endoscopes or other endoscopic instruments. The devices disclosed herein comprise multiple features that address critical issues with the successful delivery of accessories onto an endoscope and address multiple shortcomings of current approaches to delivering accessories on to an endoscope, especially with respect to delivery onto the distal or working end of an endoscope. One or more of these features may be used in an endoscope accessory loader, depending on the specific accessory being loaded onto the endoscope. Additionally, the endoscope accessory loader may be used on a stand-alone basis or in connection with the use of a delivery system designed for use with an accessory.

Suitable accessory devices may include coupler devices used for covering and sealing a portion of the working end of an endoscope. The coupler devices protect the endoscope and its components, particularly the endoscope elevator, to reduce the risk of debris, fluid and other matter ending up in the elevator and behind the elevator and the working channel that potentially cause infection. Other coupler devices not only cover and seal the working end of an endoscope, but also preserve visualization for the endoscope when in contact with fluid, debris and tissue. Coupler devices suitable for use with the loading devices described herein are found in U.S. Pat. Nos. 10,506,918, 10,856,724 and 10,101,574, the complete disclosures of which are incorporated herein by reference for all purposes.

In one aspect, a loading device includes a main body or base element having a proximal portion for receiving an endoscopic device, such as an endoscope, and a distal portion for receiving an accessory device. The main body further includes a channel or passage extending along the proximal and distal portions. The loading device allows a healthcare professional to advance the endoscope and the accessory device into the channel to attach them to each other.

The main body or base element of the loading device allows an operator to stabilize the delivery area of the endoscope in a predictable, consistent manner. For example, the main body allows the operator to hold all, or a portion of, the endoscope, preferably the portion close to the distal end of the endoscope, in the channel of the loading device, thereby stabilizing the endoscope prior to delivery of the accessory. In embodiments, this aspect of the technology allows the endoscope to be stabilized either by the users hand or through resting the loader on a fixed surface, like a table top or other predictable, stable surface. This feature not only helps create a more stable delivery circumstance, but it also helps offset a tendency for the endoscope to flop to one side or another due to the torque created by the controller area of the endoscope on the distal end. The additional weight of cabling, valves and other aspects of the endoscopic system provide more weight than other areas of the endoscope, making its working end prone to flop or shift to one side as one attempts to maneuver the endoscope for the delivery of an accessory onto the endoscope. This embodiment of the device provides a way to stabilize and avoid shifting of the distal end of the endoscope during delivery of an accessory on to the endoscope.

In embodiments, the loading device further includes a fixation element for positioning and securing the endoscope in place relative to the loading device. In one such embodiment, the proximal portion of the loading device further includes first and second portions configured to move relative to each other between open and closed positions. In the open position, the first and second positions are configured to receive the working end of the endoscope. In the closed position, the first and second portions secure the distal or working end of the endoscope in place relative to the loading device.

In one such embodiment, the fixation element may include a hinge that couples upper and lower portions of the main body to each other and allows the operator to move these portions into a closed position around the working end of the endoscope. This fixation element stabilizes the distal end of the endoscope to prevent it from flexing and articulating during delivery of an accessory on to the endoscope. Current approaches to delivery that involve holding the distal end of the endoscope in one hand and pushing the accessory onto the endoscope with another hand are challenging because the distal end of most endoscopes are designed to be flexible and to articulate. Thus, these distal ends typically deflect as force is applied to push an accessory on to the endoscope. This embodiment addresses this issue by providing a stable base that minimizes or otherwise does not deflect, thereby significantly improving accessory delivery.

The fixation element may alternatively (or in conjunction with the hinge) include a compression fit holder, a slide through element that surrounds the distal end of the endoscope, a snap on temporary holder, an adaptor that fits over the endoscope and then attaches (e.g., temporarily) to the endoscope, or any other element that can temporarily stabilize the distal end of the endoscope for accessory delivery.

In embodiments, the proximal portion of the loading device further includes a locking feature. The locking feature is configured to engage a mating feature to lock the proximal portion of the loading device in the closed configuration.

In embodiments, the loading device further includes a positioning element coupled to the main body. The positioning element allows the user to confirm that the orientation and/or position of the endoscope in the loading device is at the correct or optimal point for predictable, successful delivery of an accessory onto the endoscope. In embodiments, the loading device also includes an element for temporarily fixing the position of the endoscope relative to the loading device after confirming the proper orientation and/or position of the endoscope relative to the loading device, so that delivery can occur without moving the endoscope.

The positioning element can take multiple forms, including a marker line or other visual indicator, a stop mechanism to push the endoscope against, a hinge with one or more interference points to make sure the endoscope is delivered properly, one or more alignment elements including on a hinge or other aspect of the loading device, such as a cut-out area to use to confirm orientation relative to a key point or element of the endoscope. The key point or element may be, for example, an endoscope camera, light source, endoscope washer, elevator region, elevator indentation, distal end or other reference point or marker to confirm orientation. The positioning element may take any form that allows for repeatable, successful positioning of the loading device relative to the endoscope to enable successful delivery. This may also include attachable, detachable or other temporary elements that can go over the endoscope, on the endoscope, alongside the endoscope and/or the endoscope accessory loader, or both to align and/or confirm proper orientation

The loading device may further include a delivery tract that guides the accessory device into the channel of the loading device. In addition, or alternatively, the loading device may include a separate delivery system for advancing the accessory device onto the loading device. The delivery tract may comprise single or multiple guide tracts that effectively position the accessory for consistent, repeatable delivery on to the endoscope. In embodiments, the delivery tract comprises a pair of guide tracks formed on the distal portion of the main body. The guide tracks may extend laterally outward from the longitudinal axis of the main body and may form a central opening for placement of the accessory device thereon. Alternatively, the accessory device or a delivery system for the accessory track may contact a feature that fits within the guide track to follow the track for more precise and predictable delivery.

The guide track(s) minimize the variable aspects of delivery currently present for the delivery of accessories where the path for delivery, the angle of delivery, the amount of force or pressure to engage in delivery and to complete delivery, as well as other variables, are not clear and therefore are not consistent from one healthcare professional to the next.

In embodiments, the loading device may include delivery confirmation elements that guide another key aspect of the delivery to affirm to the user that delivery is complete. In embodiments, the delivery confirmation elements may include a ramp on the loading device and/or an indicator, such as a click, a bump or other element that provides an audible signal and/or a change in tactile feel to affirm to the operator that delivery is complete and the user does not need to push any further to achieve successful delivery.

In embodiments, the loading device may include a rib, cross-member or other element that may prevent further pushing or other application of force to indicate to the user in an additional or different way than an audible or tactile signal, that the delivery of the accessory is completed and that the accessory is fully-delivered on to the endoscope. Failure to fully deliver accessories is a known issue with accessory related injury, with multiple adverse event notices to the FDA, as well as multiple warnings from endoscope manufacturers concerning this issue. This allows a healthcare worker with gloves on to deliver an accessory on to an endoscope in a consistent, repeatable manner without wondering if delivery was completed as instructed and intended (i.e. did they push too far or not far enough).

In embodiments, the loading device further includes one or more brace members. In embodiments, the brace members are formed on the main body of the loading device. In embodiments, the brace members extend laterally outward from the longitudinal axis defined by the channel of the loading device and serve to provide a support for the loading device. For example, the brace members may be positioned against a suitable surface, such as a table, to stabilize the loading device while the accessory device is delivered onto the endoscope.

In another aspect, a system for coupling an accessory device to an endoscopic device comprises a loading device having a main body having a proximal portion for receiving an endoscopic device, such as an endoscope, and a distal portion for receiving an accessory device. The system further includes a delivery device for advancing the accessory device through the distal portion to couple the accessory device to the endoscopic device.

In embodiments, the loading device includes a channel or passage extending along the proximal and distal portions and one or more fixation elements for holding the endoscopic device in a substantially fixed position relative to the main body.

In embodiments, the system may further comprise a removal element for removing the accessory device from the delivery device. The removal element may comprise a second fixation element on the main body of the loading device for securing the accessory device to the loading device. The second fixation element may be positioned to secure the accessory device to the loading device when the accessory device has been coupled to the endoscopic device.

In embodiments, the system further comprises an introducer configured to engage with an element at a distal end of the endoscopic device. The introducer serves to further align the endoscope with the delivery element and the accessory device. In one such embodiment, the introducer comprises a rod that aligns with the endoscope's instrument channel or other element at the distal end of the endoscope. The rod may, for example, be sized to extend into the instrument channel such that the delivery device is precisely aligned with the endoscope.

In embodiments, the system may also include expander elements configured to expand the distal end of the accessory device or the delivery device temporarily during delivery to enlarge the end of the accessory device as it travels onto the endoscope. The expander element(s) may be removed individually, or they may be part of another system, i.e., attachable/detachable with the endoscope accessory loader or a delivery device that works with the endoscope accessory loader (or as a standalone element to aid in delivery).

In embodiments, the device may include a removal element that can be attached to the device or can be disposed on a hinge or other elements by which it can be placed over or in contact with the accessory device. This element allows any delivery system to be removed without migration or movement of the accessory device while the delivery system is removed. Additionally, this element provides an additional point of confirmation that the accessory device is pushed far enough on to the end of the endoscope. Otherwise, the removal element would not fit properly in place.

The elements of the loading device may form a single, integrated device, or they may be removably coupled to each other, e.g., snapped together, such that a user can select which elements are most important to the user. The elements can snap together individually or along a backbone, rod or connector element. The order of the elements may vary depending on the endoscope upon which the accessory is being delivered.

The loading device and all of the related elements described herein may be made of any material compatible with the delivery of an accessory device onto an endoscope. This includes, but is not limited to, metals such as nitinol, stainless steel, cobalt, chromium and other metals, and polymers that may include, but are not limited to polypropylene, urethanes, high and low density polyethelenes, silicone materials, PVC, PVDF, and any other polymer or elastic material suitable for this application.

The loading device may be sterile or non-sterile. It may be single-use, preferably, but in embodiments, also may be reusable. In embodiments, the loading device may be autoclavable, while other embodiments may be packaged and sterilized.

In embodiments, the loading device may include a colorant or marker that changes colors when sterilized to confirm or signal a sterilization process or reprocessing or autoclave process has occurred.

The loading device may contain one or more latches, locks or other fixation elements to temporarily fixate different aspects of the endoscope accessory loader during various steps in the accessory delivery process.

The endoscope accessory loader innovation is applicable to multiple devices that are delivered on to an endoscope or other endoscopic instrument, including, without limitation, devices used for aiding in tissue dissection (such as hollow caps with various edges), bleeding control (such as variceal banding devices), removable caps to provide more direct access to clean certain areas on an endoscope, disposable endoscopic shields that seal the distal end of the endoscope, hollow distal caps, colon lens, energy lens, biopsy device, EMR caps, tissue removal caps used in Barrett's esophagus procedures, and any other accessory that involves placement on or over the distal or working end of an endoscope.

The loading device may be made in any manner suitable for its intended use, which may include, without limitation, through molding, 3d printing, extruding, gluing together or assembling multiple components.

In another aspect, a method for coupling an accessory device to an endoscope or other endoscopic device comprises inserting the endoscopic device into a channel of a loading device and securing the endoscopic device therein. The accessory device is advanced through a distal end of the channel and secured to the distal end of the endoscopic device. Alternatively, the loading device may be advanced while the accessory device is held in position.

In embodiments, the method further comprises aligning an element of the distal end of the endoscopic device with a longitudinal position within the channel and/or with an orientation (e.g., angle) relative to the endoscopic device. This alignment may be achieved with any one of a marker line or other visual indicator, a stop mechanism to push the endoscope against, a hinge with one or more interference points to make sure the endoscope is delivered properly, one or more alignment elements at any point including on a hinge or other aspect of the loader, such as a cut-out area to use to confirm orientation relative to a key point on the endoscope, such as an endoscope camera, light source, endoscope washer, elevator region, elevator indentation, distal end or other reference point or marker to confirm orientation.

In embodiments, the endoscopic device may be secured to the loading device by moving first and second portions of the main body into a closed position. In a preferred embodiment, the first portion is rotated about a hinge relative to the second position into a closed position around the distal end of the endoscopic device.

In embodiments, the method further comprises aligning the endoscopic device with one or more guide tracks on a proximal portion of the loading device. The method may further comprise attaching the accessory device to a delivery device and advancing the delivery device along the one or more guide tracks into the channel of the loading device. The delivery device may be advanced until the accessory device engages the distal end of the endoscopic device.

In embodiments, the method further comprises gripping a proximal end of the loading device while the delivery device is advanced into the channel.

In embodiments, the method further comprises stabilizing the loading device by positioning one or more brace members against a surface

In embodiments, the method further comprises engaging the accessory device with one or more delivery confirmation elements, such as a click, a bump or other element that provides an audible signal and/or a change in tactile feel to affirm to the operator that delivery is complete and the user does not need to push any further to achieve successful delivery. In embodiments, the loading device may include a rib, cross-member or other element that may prevent further pushing or other application of force to indicate to the user in an additional or different way than an audible or tactile signal, that the delivery of the accessory is completed and that the accessory is full-delivered on to the endoscope.

In embodiments, the method may further comprise engaging an element on a distal end of the endoscopic device with an introducer to align the accessory device with the endoscopic device. The introducer may, for example, comprise a rod or other longitudinal element configured to advance into a channel in the endoscopic device

In embodiments, the method may further comprise expanding a proximal end portion of the accessory device to an outer dimension larger than an outer dimension of the distal end of the endoscopic device. This allows the operator to couple the accessory device to the endoscopic device by advancing the proximal end portion of the accessory device over the distal end of the endoscopic device. The expander elements can then be removed such that the proximal end portion of the accessory device then conforms, and secures to, the distal end of the endoscopic device.

It is to be understood that both the foregoing general description and the following detailed description are exemplary and explanatory only and are not restrictive of the disclosure. Additional features of the disclosure will be set forth in part in the description which follows or may be learned by practice of the disclosure.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1 is a perspective top view of a loading device;

FIG. 2 is a perspective bottom view of the loading device of FIG. 1;

FIG. 3 is a front view of the loading device of FIG. 1 in an open position;

FIG. 4 is a side view of the loading device in a closed position;

FIG. 5 is a top view of the loading device;

FIG. 6 is a bottom view of the loading device;

FIG. 7 is a perspective view of one embodiment of a loading device in use with an illustrative endoscope;

FIG. 8 is a perspective view of another embodiment of a loading device in use with an illustrative endoscope;

FIG. 9 depicts a healthcare professional using an insertion tool to prepare to attach a coupler device to the working end of an endoscope;

FIG. 10 depicts a healthcare professional using an insertion tool to prepare to attach a coupler device to the working end of an endoscope using a bracing element of the loading device;

FIG. 11 is a diagrammatic side view of the exemplary loading device of FIG. 1 resting on a flat surface with a bracing element;

FIG. 12 is a top view of a distal portion of a loading device;

FIG. 13 is a perspective view of a system for loading an accessory device onto an endoscopic instrument, illustrating a grip hinge and a removal hinge in a closed position;

FIG. 14 is a side view of the system of FIG. 13, illustrating the grip hinge in a closed position;

FIG. 15 is a side view of the system of FIG. 13, illustrating the grip hinge in a closed position;

FIG. 16 is a perspective view of the system of FIG. 13, illustrating the grip hinge in a closed position;

FIG. 17 is a perspective view of the system of FIG. 13 without the delivery device with the grip hinge in a closed position; and

FIG. 18 is a perspective view of the system of FIG. 13 without the delivery device with the grip hinge in an open position.

### DETAILED DESCRIPTION

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. In some instances, well-known structures and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

While the following description is primarily directed to a disposable companion or coupler device for use with a reusable optical image endoscope, it should be understood that the features of the presently described loading systems and devices may be readily adapted for use with a variety of reusable or disposable endoscopes, endoscopic instruments and other devices and with multiple devices that are delivered on to an endoscope or other endoscopic instrument, including, without limitation, devices used for aiding in tissue dissection (such as hollow caps with various edges), bleeding control (such as variceal banding devices), removable caps to provide more direct access to clean certain areas on an endoscope, disposable endoscopic shields that seal the distal end of the endoscope, hollow distal caps, colon lens, energy lens, biopsy device, EMR caps, tissue removal caps used in Barrett's esophagus procedures, and any other accessory that involves placement on or over the distal or working end of an endoscope.

The term “endoscope” refers generally to any scope used on or in a medical application, which includes a body (human or otherwise) and includes, for example, a laparoscope, duodenoscope, arthroscope, colonoscope, bronchoscopes, enteroscope, cystoscope, laparoscope, laryngoscope, sigmoidoscope, thoracoscope, cardioscope, and saphenous vein harvester with a scope, whether robotic or non-robotic.

When engaged in remote visualization inside the patient's body, a variety of scopes are used. The scope used depends on the degree to which the physician needs to navigate into the body, the type of surgical instruments used in the procedure and the level of invasiveness that is appropriate for the type of procedure. For example, visualization inside the gastrointestinal tract may involve the use of endoscopy in the form of flexible gastroscopes and colonoscopes and specialty duodenum scopes with lengths that can run many feet and diameters that can exceed 1 centimeter. These scopes can be turned and articulated or steered by the physician as the scope is navigated through the patient. Many of these scopes include one or more working channels for passing and supporting instruments, fluid channels and washing channels for irrigating the tissue and washing the scope, insufflation channels for insufflating to improve navigation and visualization and one or more light guides for illuminating the field of view of the scope.

Smaller and less flexible or rigid scopes, or scopes with a combination of flexibility and rigidity, are also used in medical applications. For example, a smaller, narrower and much shorter scope is used when inspecting a joint and performing arthroscopic surgery, such as surgery on the shoulder or knee. When a surgeon is repairing a meniscal tear in the knee using arthroscopic surgery, a shorter, more rigid scope is usually inserted through a small incision on one side of the knee to visualize the injury, while instruments are passed through incisions on the opposite side of the knee. The instruments can irrigate the scope inside the knee to maintain visualization and to manipulate the tissue to complete the repair

Other scopes may be used for diagnosis and treatment using less invasive endoscopic procedures, including, by way of example, but not limitation, the use of scopes to inspect and treat conditions in the lung (bronchoscopes), mouth (enteroscope), urethra (cystoscope), abdomen and peritoneal cavity (laparoscope), nose and sinus (laryngoscope), anus (sigmoidoscope), chest and thoracic cavity (thoracoscope), and the heart (cardioscope). In addition, robotic medical devices rely on scopes for remote visualization of the areas the robotic device is assessing and treating.

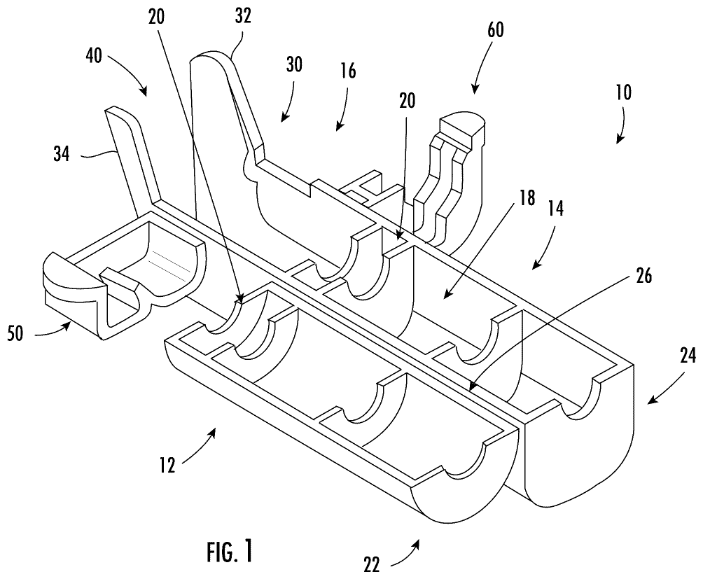

FIG. 1 illustrates a loading device 10 for an accessory device that includes a main body 12 having a proximal portion 14 and a distal portion 16. Loading device 10 may be made using a single-mold construction, though alternative designs are envisioned such as multiple molded parts, additive manufacturing techniques (e.g., 3D printing) and the like. A channel or passage 18 is formed along and defines a longitudinal axis of loading device 10 that extends through the central portions of proximal portion 14 and distal portion 16. Channel 18 is configured to receive an endoscopic device, such as an endoscope or the like.

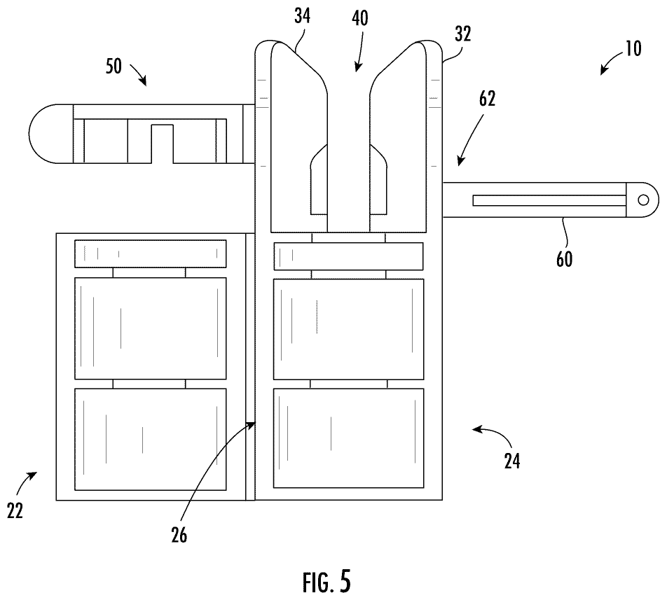

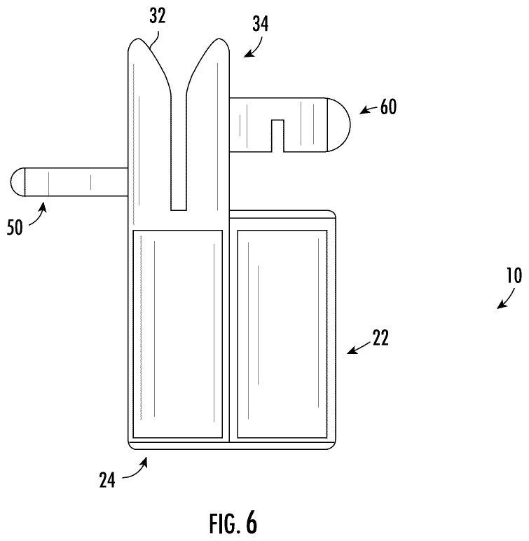

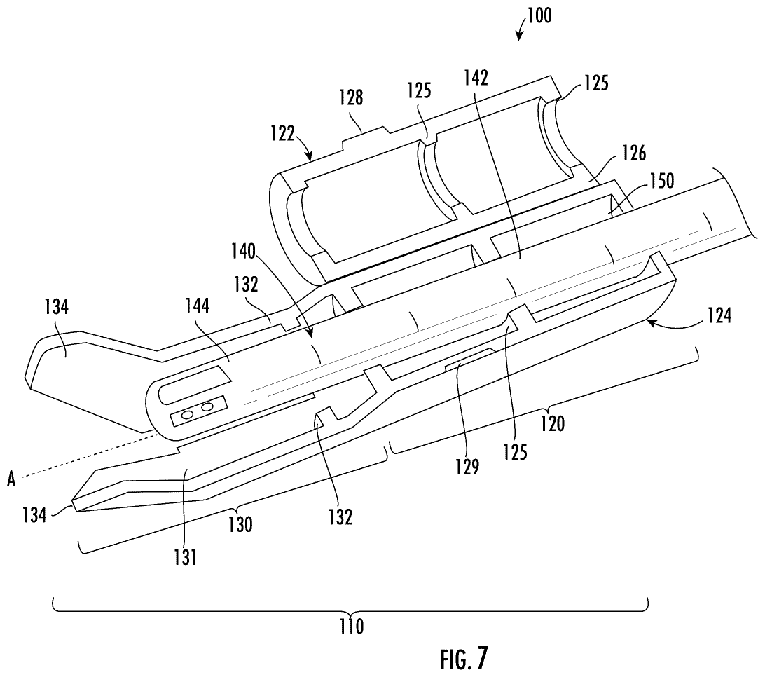

Proximal portion 14 of main body 12 includes first and second portions 22, 24 that are movable between an open position (FIG. 5) and a closed position (FIG. 6). The open position allows the operator to easily insert an endoscopic device within channel 18 and the closed position allows the operator to secure the endoscopic device to loading device 10. In one embodiment, first and second portions 22, 24 are connected by a hinge 26 therebetween. In use, this clamshell hinge configuration allows first and second portions 22, 24 to pivot relative to each other to close around a distal end portion of an endoscopic device (see, for example, FIG. 7). In the closed position, proximal portion 14 may be easily gripped by a healthcare professional for stabilizing loading device 10. As an example of an alternative to a clamshell hinge, a component could also snap on or affixed and/or articulated by other means.

Loading device 10 may include one or more alignment elements 20 for aligning the endoscopic device along the longitudinal axis or the center of channel 18. Alignment elements 20 may comprise protrusions or ribs or solid surfaces formed on inner surfaces of one or both of first and second portions 14, 16 to further stabilize the endoscopic device within channel 18. In embodiments, alignment elements 20 may be ribs extending across channel 18 that include a cut-out or other recess for receiving the shaft of the endoscopic device, though any suitable structures or designs configured to promote stability of the endoscopic device may be employed.

Distal portion 16 of loading device 10 includes a base portion 30 configured to receive and support the working end of an endoscopic device. Base portion 30 houses the working end of the endoscopic device, but does not cover the upper half of the device. This allows a clinician to access the working end to insert an accessory device thereon (discussed below).

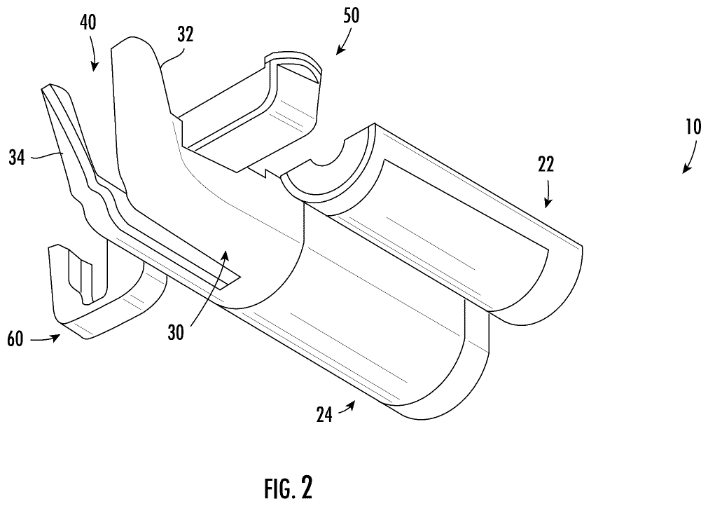

Distal end portion 16 further includes a pair of guide tracks 32, 34 formed on the distal end of base portion 30. Guide tracks 32, 34 preferably have a central opening 40 therebetween (see FIG. 2) and extend laterally outward from the longitudinal axis of loading device 10. Guide tracks 32, 34 facilitate the use of an insertion tool by a clinician to install an accessory device onto the working end of the endoscopic instrument. Guide tracks 32, 34 may extend at any suitable angle, but preferably extend laterally outward sufficiently to provide easy access for the accessory device.

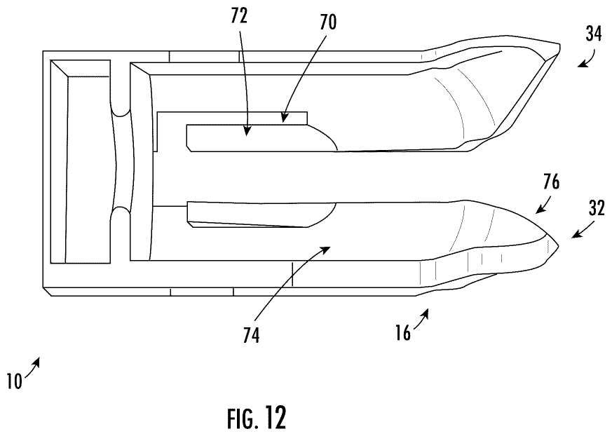

FIG. 12 illustrates an alternative embodiment of distal end portion 16 of a loading device 10. In this embodiment, distal end portion 16 includes a cut-out 70 that forms a ramp 72 on a proximal portion of guide track 34. Distal end portion 16 may include a similar cut-out on guide track 32 (not shown). Ramps 72 serve to deflect downwards (into the page) upon delivery of the accessory device into loading device 10. Ramps 72 will then snap upwards back into their original position after the expander elements (discussed below) pass in the proximal direction. This provides more flexibility to guide tracks 32, 34 to allow the expander elements to pass through and then expand the proximal end of accessory device (also discussed below).

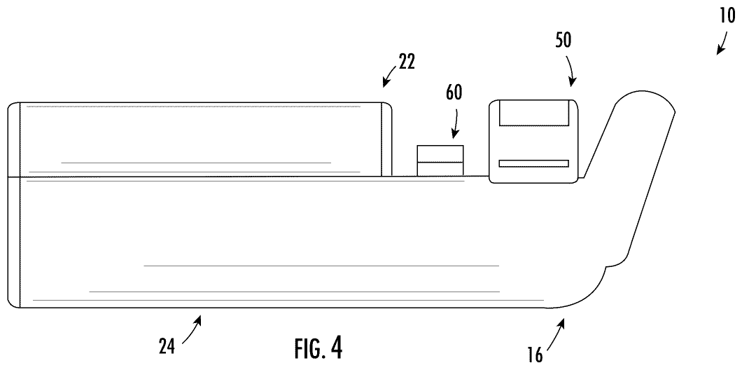

Guide tracks 32, 34 also have a different configuration in this embodiment than in the embodiment shown in FIGS. 1-11. As shown, guide tracks 32, 34 include proximal portions 74 that are substantially linear and substantially parallel to the longitudinal axis of loading device 10. The proximal portions 74 may also have an arcuate inner surface that curves inwardly (i.e., semi-concave) in the lateral direction. Guide tracks further include distal portions 76 that bend or curve in two dimensions relative to the proximal portions. Th distal portions 76 curve upwardly (i.e., out of the page) relative to proximal portions. In addition, the distal portions 76 curve laterally outward from the longitudinal axis of loading device 10. This configuration facilitates the guiding of a delivery device and the accessory device along guide tracks 32, 34 and into loading device 10.

Of course, other configurations are envisioned. For example, distal end portion 16 may include a ramp for guiding the accessory device into channel 18. The ramp may have curved inner surface that allows the accessory device to be guided along this surface into channel 18. Alternatively, distal end portion 16 may have a closed end with a distal opening for passing accessory device therethrough.

Referring again to FIG. 1, distal portion 16 further includes a positioning element or alignment indicator 50 that allows the user to confirm that the orientation of the endoscopic instrument in device loader 10 is at the correct or optimal position for predictable, successful delivery of an accessory onto the instrument. In embodiments, alignment indicator 50 may be an arrow, color, number, letter, word, protrusion, surfaced feature or any other suitable marking for visualizing alignment of a given component of the working end of the instrument may be used. For example, alignment indicator 50 may be used to align the camera lens or the working channel of an endoscope. The healthcare provider positions one of these elements at the alignment indicator, which ensures that the endoscope is at the proper longitudinal position within loading device 10.

Of course, this positioning element can take multiple forms, including use of a marker line, a stop to push the endoscope against, a hinge with one or more interference points to make sure the endoscope is delivered properly, one or more alignment elements at any point including on a hinge or other aspect of the loader, such as a cut-out area to use to confirm orientation relative to a key point on the endoscope, such as an endoscope camera, light source, endoscope washer, elevator region, elevator indentation, distal end or other reference point or marker to confirm orientation. The positioning element may take any form that allows for repeatable, successful positioning of the endoscope loader relative to the endoscope to enable successful delivery. This may also include attachable, detachable or other temporary elements that can go over the endoscope, on the endoscope, alongside the endoscope and/or the endoscope accessory loader, or both to align and/or confirm proper orientation

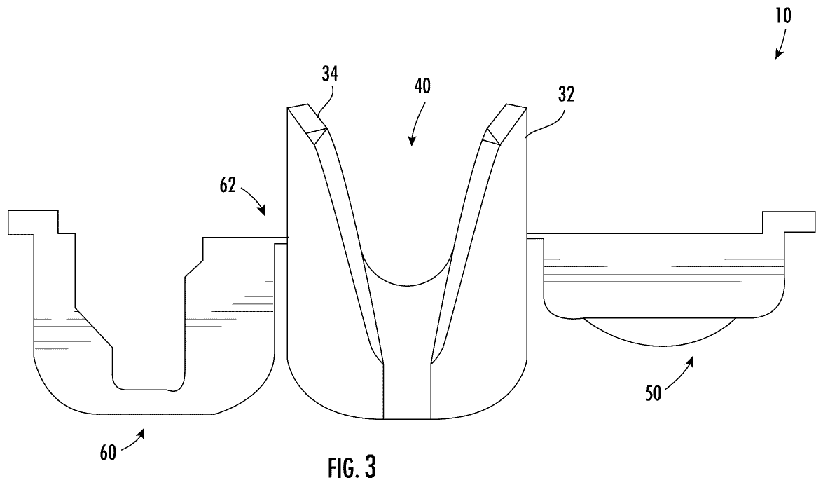

Distal portion 16 may further include a fixation element 60 for securing the accessory device to loading device 10 and/or the endoscopic device. Fixation element 60 extends laterally outward from distal portion 16 and is rotatably coupled thereto by a hinge 62 or other suitable rotational element (see FIG. 3). Fixation element 60 allows any delivery system to be removed without migration or movement of the accessory device while the delivery system is removed (as discussed below). Additionally, fixation element 60 provides an additional point of confirmation that the accessory is pushed far enough on to the end of the endoscope so that it is fit properly in place.

FIG. 7 illustrates an exemplary embodiment of a loading device 100 in use with an endoscopic device, such as an endoscope 140. As shown, loading device 100 may include a main body 110 having a proximal portion 120 and a distal portion 130. A channel or passage 150 is formed along and defines a longitudinal axis ‘A’ of loading device 100 that extends through the central portions of proximal portion 120 and distal portion 130. Channel 150 is configured to receive an endoscope 140 having an elongated portion 142 and a working end 144.

Proximal portion 120, which typically supports a portion of the elongated portion 142 of a given endoscope 140, includes an upper half 122 and a lower half 124. Upper half 122 and lower half 124 are connected by a hinge 126 therebetween. In use, this clamshell hinge configuration allows upper half 122 and lower half 124 to move towards each other to close around the elongated portion 142 of an endoscope 140. In the closed position, proximal portion 120 may be easily gripped by a clinician for stability of loading device 100.

Protrusions 125 are formed on inner surfaces of one or both of upper half 122 and lower half 124 to further stabilize endoscope 140 within channel 150. In embodiments, protrusions 125 may be ribs, though any suitable structures or designs configured to promote stability of endoscope 140 may be employed. A locking feature 128 may be formed on a portion of upper half 122, while a complementary mating feature 129 may be formed on lower half 124. Upon closing, locking feature 128 will engage mating feature 129 to lock the proximal portion 120 in the closed configuration. In embodiments, the locking feature may be a snapping feature, however, other suitable locking features, such as, for example, cantilever hooks, cantilever holes, window snaps, annular snaps, leaf-spring snaps, ball and socket, post and dome, compression hooks, compression traps, compression beams, bayonet finger snaps, torsion snaps, integral spring tabs, spring plugs, spring clips, snap slides, and quick release fasteners may be used.

Distal portion 130 of loading device 100 includes a base portion 131 configured to receive and support the working end 144 of an endoscope 140. Base portion 131 houses the working end 144 of endoscope 140, but does not cover the upper half of working end 144. This allows a clinician to access the working end 144 to insert a coupler device thereon. Protrusions 132 are formed on base portion 131 to further stabilize endoscope 140 within channel 150. In embodiments, protrusions 132 may be ribs, though any suitable structures or designs configured to promote stability of endoscope 140 may be employed.

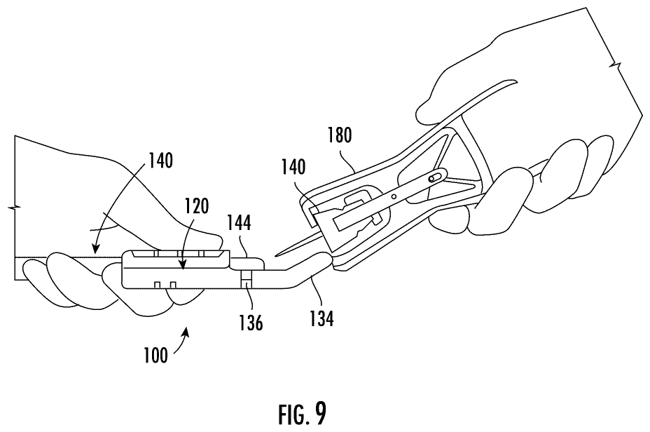

Distal end portion 130 further includes a pair of guide tracks 134 formed on the distal end of base portion 131. Guide tracks 134 facilitate the use of an insertion tool by a clinician to install a coupler device onto working end 144 of endoscope 140 using loading device 100. Guide tracks 134 may extend at any suitable angle. Guide tracks 134 further includes an alignment indicator (not shown). In embodiments, the alignment indicator may be an arrow, but any suitable marking for visualizing alignment of a given component of working end 144 of endoscope 140 may be used. Use of loading device 100 in combination with an insertion tool or delivery device will be further described below in connection with FIG. 9.

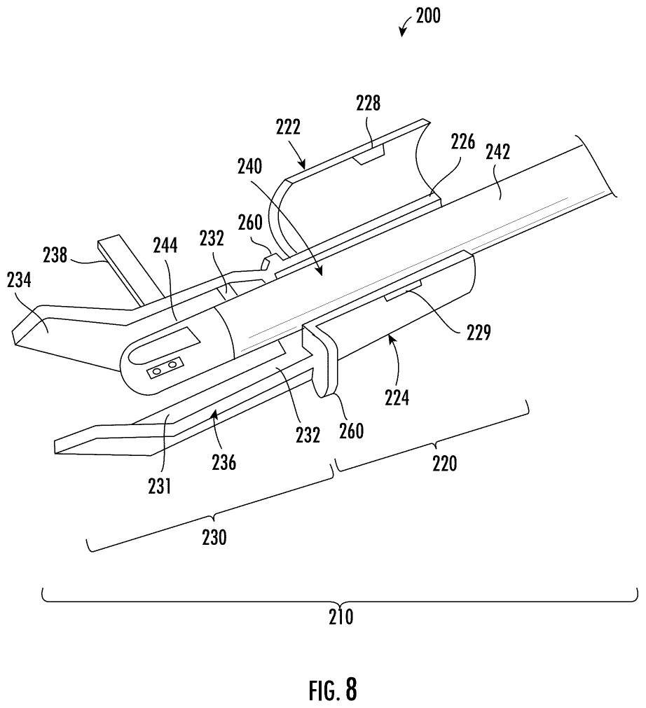

FIG. 8 depicts a perspective view of a second exemplary embodiment of a loading device 200 in use with a duodenoscope 240. Loading device 200 may include a main body 210 having a proximal portion 220 and a distal portion 230. A channel is formed along a longitudinal axis of loading device 200 that extends through the central portions of proximal portion 220 and distal portion 230. The channel is configured to receive an endoscope 240 having an elongated portion 242 and a working end 244.

Proximal portion 220, which typically supports the elongated portion 242 of a given endoscope 240, includes an upper half 222 and a lower half 224. Upper half 222 and lower half 224 are connected by a hinge 226 therebetween. In use, this clamshell hinge configuration allows upper half 222 and lower half 224 to move towards each other to close around the elongated portion 242 of an endoscope 240. In the closed position, proximal portion 220 may be easily gripped by a clinician for stability of loading device 200. Protrusions or other surface features may be formed on inner surfaces of one or both of upper half 222 and lower half 224 to further stabilize endoscope 240 within channel 250. In embodiments, protrusions 225 may be ribs, though any suitable structures or designs configured to promote stability of endoscope 240 may be employed.

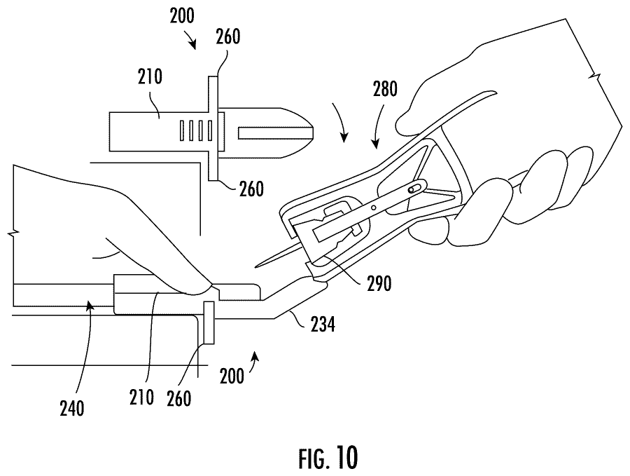

A locking feature 228 may be formed on a portion of upper half 222, while a complementary mating feature 229 may be formed on lower half 224. Upon closing, locking feature 228 will engage mating feature 229 to lock the proximal portion 220 in the closed configuration. Loading device 200 further includes brace members 260. In embodiments, brace members 260 are formed on each side of the distal end portions of the lower half of proximal portion 220. In use, brace members 260 function to stabilize loading device 200 against or on top of a given surface. Thus, brace members 260 may extend towards any desired direction and at any desired angle to complement the structure or surface being used to stabilize loading device 200. In embodiments, brace members 260 extend downwards in a direction that is substantially perpendicular to longitudinal axis A. Examples of how brace members 260 of loading device 200 function in use are further described below in connection with FIGS. 9 and 10.

Distal portion 230 of loading device 200 includes a base portion 231 configured to receive and support the working end 244 of an endoscope 240. Base portion 231 houses the working end 244 of endoscope 240, but does not cover the upper half of working end 244. This allows a clinician to access the working end 244 to insert a coupler device thereon. Protrusions 232 are formed on base portion 231 to further stabilize endoscope 240 within channel 250. In embodiments, protrusions 232 may be ribs, though any suitable structures or designs configured to promote stability of endoscope 240 may be employed. Distal end portion 230 further includes a pair of guide tracks 234 formed on the distal end of base portion 231. Guide tracks 234 facilitate the use of an insertion tool by a clinician to install a coupler device onto working end 244 of endoscope 240 using loading device 200.

Guide track 234 may further include an alignment indicator 236 on a first side of guide track 234. In embodiments, alignment indicator 236 may be an arrow, but any suitable marking for visualizing alignment of a given component of working end 244 of endoscope 240 may be used. In embodiments, loading device 200 may include a second alignment indicator 238 on an opposing side of guide track 234 with respect to alignment indicator 236. Use of loading device 200 in combination with an insertion tool will be further described below in connection with FIGS. 9 and 10.

FIG. 9 depicts a system and method for delivering an accessory device 190 onto an endoscope 140. An illustrative delivery device or insertion tool 180 is shown having an illustrative coupler device 190 for attachment to the working end of an endoscope secured thereto. An exemplary insertion tool 180 is described in greater detail, for example, in commonly owned PCT Publication No. 2020/0390319 A1, the entirety of which is incorporated by reference herein. Coupler device 190 may, for example, be used for covering and sealing a portion of the working end of an endoscope. The coupler devices protect the endoscope and its components, particularly the endoscope elevator, to reduce the risk of debris, fluid and other matter ending up in the elevator and behind the elevator and the working channel that potentially cause infection. Coupler devices suitable for use with the loading devices described herein are found in U.S. Pat. Nos. 10,506,918, 10,856,724 and 10,101,574, the complete disclosures of which are incorporated herein by reference for all purposes.

In operation, the healthcare professional places the endoscope into the central channel of loading device 100 and closes proximal portion 120 of loading device 100 around the endoscope 140, ensuring that the alignment indicators 136 are aligned with the lens or other suitable element of working end 144 of endoscope 140. In this closed configuration, proximal portion 120 of loading device 100 provides a highly ergonomic and easy-to-grip surface while also ensuring that endoscope 140 is both secure and stable. The clinician may grip loading device 100 using a first hand. Next, the clinician may use their second hand to grip insertion tool 180. The clinician may then use guide tracks 134 of loading device 100 to easily guide insertion tool 180 towards the working end 144 of the endoscope 140 to insert and secure coupler device 190 to the working end 144 of endoscope 140. Insertion tool 180 may be any suitable or desired insertion tool.

In some embodiments, the clinician may use one or more delivery confirmation elements (described above) to ensure that the accessory device 190 is properly positioned against the endoscope 140. In addition, or alternatively, insertion tool 180 may include an introducer, such as a rod or other tube, extending proximally from insertion tool 180. The introducer is configured to contact and engage an element of the working end of endoscope, such as an instrument channel. This engagement ensures that the accessory device 190 is properly aligned with the endoscope 140.

In addition, the system may include one or more expandable elements (not shown) configured to expand the proximal end of the accessory device. In certain embodiments, the method may further comprise expanding a proximal end portion of the accessory device 190 to an outer dimension larger than an outer dimension of the distal end of the endoscope 140. This allows the operator to couple the accessory device to the endoscopic device by advancing the proximal end portion of the accessory device over the distal end of the endoscopic device. The expander elements can then be removed such that the proximal end portion of the accessory device then conforms, and secures to, the distal end of the endoscopic device.

Once the accessory device 190 is suitable attached to the endoscope 140, the clinician may remove insertion tool 180. In certain embodiments, fixation element 60 (see FIG. 1) may be rotated about hinge 62 into a closed position to secure accessory device 190 to loading device 100. Fixation element 60 allows the insertion tool 180 to be removed without migration or movement of the accessory device.

FIG. 10 depicts a perspective view of a clinician using a delivery device or insertion tool 280 to attach a coupler device 290 to the working end of an endoscope 240 utilizing the exemplary loading device 200 of FIG. 8. In FIG. 10, two methods of using loading device 200 using the edge of a table or table-like surface are illustrated. In each illustrated example, brace members 260 and main body 210 of loading device 200 form a substantially right angle, thereby allowing a clinician to utilize an edge of a table or other surface to stabilize loading device 200. The substantially right angle formed by main body 210 and at least one of brace members 260 may be flush against the right angle of any table-like surface to further enhance stability of loading device 200, and endoscope 240 contained therein.

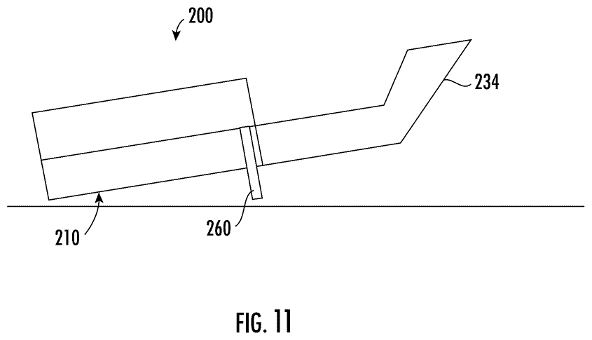

As shown in FIG. 10, loading device 200 may rest flat on a table-like surface, or may be turned on its side, allowing for a clinician to side load a coupler device 290 onto endoscope 240. This configuration still allows for at least one brace member 260 to be used for stabilization of loading device 200. Alternatively, as shown in FIG. 11, a clinician may choose to position angle brace members 260 onto a top surface of a table or other table-like structure to stabilize loading device 200.

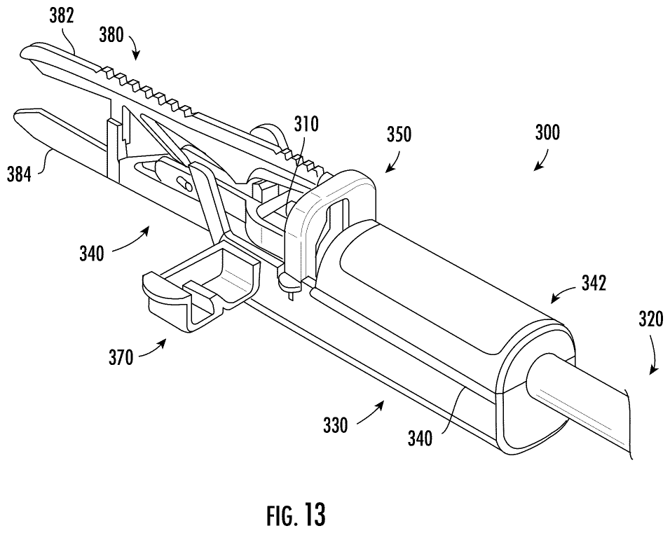

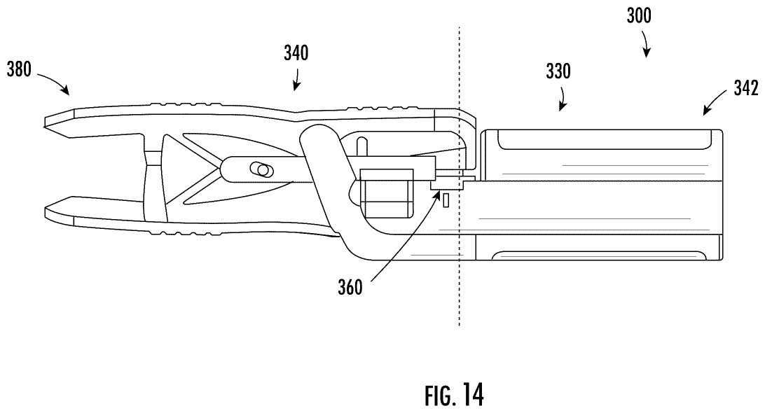

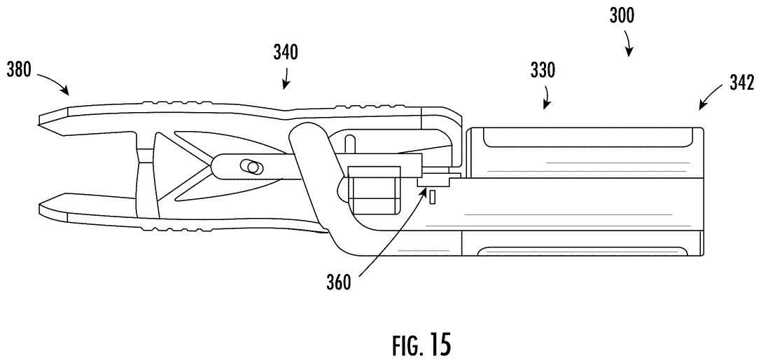

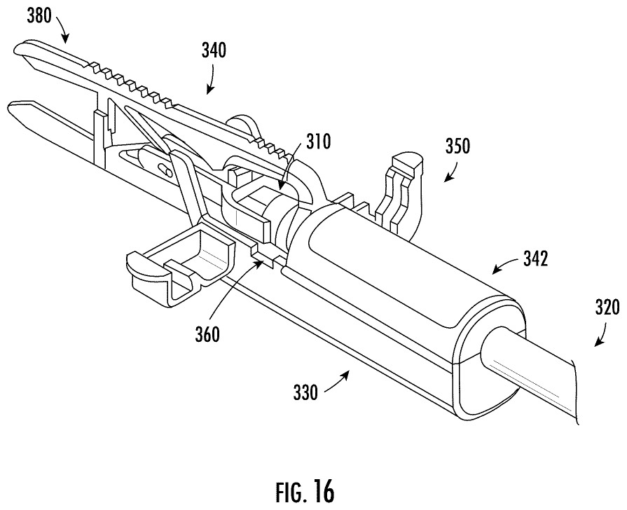

FIGS. 13-18 illustrate another embodiment of a system 300 for loading an accessory device 310 onto an endoscope 320. As shown, system 300 includes a loading device 330 and a delivery device 340. Loading device 330 includes a grip hinge 342, a removal hinge 350 and an alignment hinge 370. FIG. 13 illustrates system 300 with grip hinge 342 and a removal hinge 350 in the closed position and delivery device 340 seated against a first rib 360 of loading device 330 (see FIG. 14). As discussed above, grip hinge 342 closes the proximal portion of loading device 330 to secure the endoscope 320 within loading device 330. Removal hinge 350 closes over the delivery device 340 and the accessory device 310 to facilitate removing delivery device 340 without removing accessory device 310 from endoscope 320 (i.e., after the accessory device has been attached to the endoscope).

Delivery device 340 includes a distal handle 380 that allows the user to grip device 340 when loading accessory device 310 into the loading device 330. In addition, distal handle 380 may include first and second portions 382, 384 that are pivotally coupled to each other to allow the user to close handle 380. Closing handle 380 releases accessory device 310 from delivery device 340. Delivery device 340 may further include a spring or other biasing mechanism 386 that maintains first and second portions 382, 384 of handle 380 in the open position to ensure that accessory device 310 does not accidently release from delivery device 340.

FIGS. 14-16 illustrate system 300 with grip hinge 342 in the closed position and removal hinge 350 in the open position. Delivery device 340 is seated against the first rib 360 of loading device 330 to ensure that accessory device 310 is properly positioned for attachment to endoscope 320.

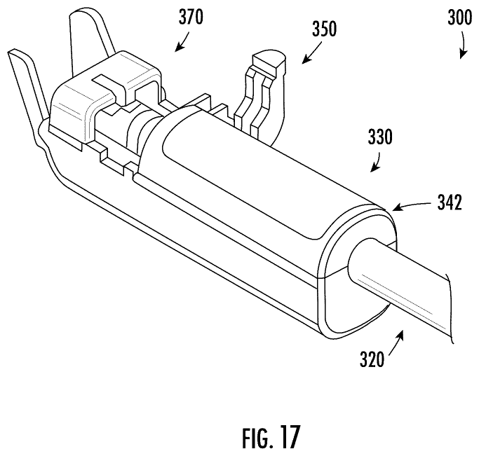

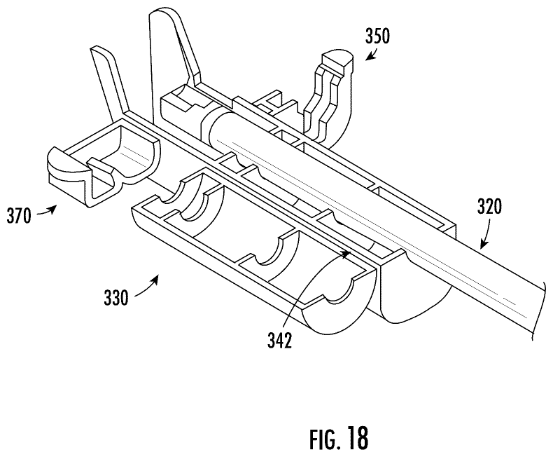

FIG. 17 illustrates system 300 with grip hinge 342 and alignment hinge 370 in the closed position and removal hinge 350 in the open position. Alignment hinge 370 serves to align the endoscope 320 in the optimal position within loading device 330, as discussed above. The accessory device 310 has not been delivered into loading device 310. FIG. 18 illustrates system 300 with grip hinge 350, alignment hinge 370 and removal hinge 342 in the open position. The accessory device has not been inserted into loading device 330.

Other embodiments will be apparent to those skilled in the art from consideration of the specification and practice of the embodiments disclosed herein. It is intended that the specification and examples be considered as exemplary only, with a true endoscope and spirit of the invention being indicated by the following claims.

What is claimed is:

1. A loading device for coupling an accessory device to an endoscopic device, the loading device comprising:
a main body having a proximal portion for receiving an endoscopic device and a distal portion for receiving an accessory device; and
a passage extending along a longitudinal axis of the main body such that the endoscopic device may be coupled to the accessory device within the passage.

2. The loading device of claim 1 further comprising a fixation element for holding the endoscope in a substantially fixed position relative to the main body.

3. The loading device of claim 1, wherein the proximal portion of the main body comprises first and second portions movable relative to each other between open and closed positions.

4. The loading device of claim 3, wherein the first and second portions hold the endoscopic device in the substantially fixed position in the closed position.

5. The loading device of claim 3, further comprising a hinge rotatably coupling the first portion to the second portion.

6. The loading device of claim 1, further comprising a positioning element coupled to the main body for aligning a portion of the endoscopic device with a position on the loading device, wherein the positioning element is configured to align the endoscopic device with the main body relative to the longitudinal axis.

7. The loading device of claim 6, wherein the positioning element extends laterally from the main body and comprises one or more visual indicators.

8. The loading device of claim 1, further comprising a guide element on the proximal portion of the main body for guiding the accessory device through the distal opening and into the passage, wherein the guide element comprises first and second guide tracks formed on the distal portion of the main body.

9. The loading device of claim 8, wherein the guide tracks are laterally spaced from the passage and extend at an angle relative to the longitudinal axis.

10. The loading device of claim 1, further comprising an indicator coupled to the main body and configured to provide an indication that the accessory device is coupled to the endoscopic device. wherein the indicator provides an audible or tactile signal when the accessory device is coupled to the endoscopic device.

11. The loading device of claim 10, wherein the indicator comprises a stop element for inhibiting further proximal movement of the accessory device relative to the endoscope.

12. The loading device of claim 1, wherein the main body comprises an inner surface and one or more protrusions extending from the inner surface to stabilize the endoscopic device within the channel.

13. The loading device of claim 1, wherein the proximal portion of the main body comprise a gripping surface.

14. The loading device of claim 3, wherein the proximal portion of the main body comprises a locking feature, the locking feature configured to lock the proximal portion of the loading device in the closed position.

15. The loading device of claim 1, wherein the loading device further comprises one or more brace member extending laterally from the main body, the brace members being configured to brace the loading device against a surface.

16. The loading device of claim 1, further comprising a fixation element for securing the accessory device to the main body, wherein the fixation element comprises a lateral extension rotatably coupled to the main body and movable between open and closed positions.

17. A system for coupling an accessory device to an endoscopic device, the system comprising:
a loading device having a main body with a proximal portion for receiving an endoscopic device and a distal portion for receiving an accessory device; and
a delivery device for advancing the accessory device into the proximal portion of the main body to couple the accessory device to the endoscopic device.

18. The system of claim 17 further comprising a passage extending along a longitudinal axis of the main body such that the endoscopic device may be coupled to the accessory device within the passage.

19. The system of claim 17 further comprising a fixation element for holding the endoscopic device in a substantially fixed position relative to the main body.

20. The system of claim 17 further comprising a removal element for removing the accessory device from the delivery device, wherein the removal element comprises a second fixation element on the main body of the loading device, the second fixation element securing the accessory device to the loading device.

21. The system of claim 20, wherein the second fixation element is positioned to secure the accessory device to the loading device when the accessory device has been coupled to the endoscopic device.

22. The system of claim 17, wherein the delivery device comprises an introducer configured to engage with an element at a distal end of the endoscopic device, wherein the introducer comprises a rod configured to advance into a channel within the endoscopic device.

23. The system of claim 17 further comprising an expander element configured to expand a proximal end portion of the accessory device, wherein the expander element expands the proximal end portion of the accessory device to an outer dimension larger than an outer dimension of the distal end of the endoscopic device.
